(19) United States
(12) Patent Application Publication
(10) Pub. No.: US 2020/0101001 A1
(43) Pub. Date: Apr. 2, 2020

(54) NOVEL SURFACTANT MIXTURE, NOVEL COMPOSITION COMPRISING SAME AND USE THEREOF IN COSMETICS

(71) Applicant: SOCIETE D'EXPLOITATION DE PRODUITS POUR INDUSTRIES CHMIQUES SEPPIC, Paris (FR)

(72) Inventors: Marie-Françoise GAYRAL CHIRAC, Viviers-les-Montagnes (FR); Jérôme GUILBOT, Castres (FR); Georges DACOSTA, Saix (FR)

(21) Appl. No.: 16/622,971

(22) PCT Filed: May 22, 2018
(86) PCT No.: PCT/FR2018/051222

§ 371 Date: Dec. 16, 2019

(30) Foreign Application Priority Data
Jun. 22, 2017 (FR) 1755694

(51) Int. Cl. A61K 8/60; A61K 8/44; A61K 8/36; A61K 8/34; A61K 8/04; A61Q 19/10; A61Q 5/02

(52) U.S. Cl. CPC A61K 8/60; A61K 8/44; A61K 8/602; A61K 8/442; A61Q 5/02; A61K 8/342; A61K 8/046; A61Q 19/10; A61K 8/36

(57) ABSTRACT

Disclosed is a method for improving the foaming properties of a cleansing formulation for topical use including glutamate-based surfactants, including a step of incorporating into the formulation an effective amount of at least one alkyl polyglycoside composition; aqueous formulation including same in combination with anionic or amphoteric surfactants; and a method for use thereof in cleansing the human or animal body.

The present invention relates to a novel process for improving the foaming properties of formulations for topical use, to novel compositions for topical use, to their use for cleansing the skin, the hair, the scalp and mucous membranes, and to a process for cleansing human and animal skin, the hair, the scalp and mucous membranes.

The present invention finds application in the human and veterinary cosmetic and dermocosmetic, dermopharmaceutical and pharmaceutical fields.

Cleansing formulations for the skin, the hair, the scalp and mucous membranes, and more generally body and hair hygiene products presented in the form of shampoos, lotions, gels or liquid soaps require the rapid formation of foam during their application to the part of the body to be cleansed. Specifically, in the mind of consumers, the rapid formation of foam constitutes one of the proofs of the cleansing efficiency. The rate of formation of the foam (or expansion time), the volume of this foam, its stability and the pleasant sensations it affords are important parameters to be taken into account in the hope of commercial success of these formulations.

Cleansing formulations comprise cleansing and foaming surfactants, whether of cationic, anionic, amphoteric or nonionic nature. These surfactants are said to be amphiphilic since they consist of a hydrophilic part (or polar head), which is water-soluble, and of a lipophilic part (hydrophobic tail) which has affinity with oils and fats. This amphiphilic structure makes it possible both to dissolve fatty soiling and to remove it by a washing operation.

Anionic surfactants are historically the surfactants that have been used for preparing foaming cleansing formulations for topical use. Sulfate-based anionic surfactants constitute a class of surfactants that is frequently used on account of their good foaming properties. These surfactants are reputed for their good cleansing power, and they also produce an airy foam which is not considered to be unpleasant when touched by consumers. However, these surfactants have the drawback of being sensitive to the degree of water hardness and to the presence of fatty soiling, which consequently leads to a reduction in the volume of foam initially generated by these formulations, but most of all to a reduction in the stability over time of this volume of foam.

To improve the stability of foams, a person skilled in the art of detergency knows that he can combine foaming surfactants with one or more additives having the effect of increasing the stiffness of the gas cells forming the foam.

Thus, the French patent application published under the number 2 439 230 discloses the use of fatty amines as foaming auxiliary, aqueous solutions of surfactants such as alkylbetaines, alkylamidobetaines, alkyl sulfates or alkyl ether sulfates. The international patent application published under the number WO 03/035794 A1 teaches of increasing the stability of a foam obtained by using at least one aliphatic hydrocarbyl phosphate monoester by adding a foam stabilizer; said foam being employed in a oil well drilling process.

Mention may also be made of the use of fatty acids as foam stabilizers, the aliphatic fatty chain of which includes from 8 to 30 carbon atoms, in mass proportions of greater than or equal to 10% by mass per 100% by mass of said cleansing formulation for topical use.

However, the foam-stabilizing additives described previously are often poorly biodegradable and are occasionally toxic, which makes them noncompliant with the new environmental requirements and regulatory provisions, and for application to the skin.

The French patent application published under the number 2 850 017 discloses a means for stabilizing cosmetic compositions in the form of foams using fatty acids in small amounts, with the proviso of obtaining this foam from an emulsion in oil-in-water form and combining with said fatty acids sugar derivatives, and more particularly alkylglucosides, said combination being described as “emulsifying”. The fatty acids used in the French patent application published under the number 2 850 017 are carboxylic fatty acids bearing a linear or branched aliphatic chain, including from 6 to 22 carbon atoms. The alkylglucosides used in the French patent application published under the number 2 850 017 are alkylglucosides having emulsifying properties, for instance the emulsifying compositions sold under the brand names Montanov™ 68 (INCI name: cetearyl alcohol and cetearyl glucoside), Montanov™ 82 (INCI name: cetearyl alcohol and coco-glucoside), Montanov™ L (INCI name: C14-22 alcohol and C12-20 alkyl glucoside). The French patent application published under the number 2 850 017 also describes mass ratios of fatty acids to alkylpolyglucosides equal to 1/1 and does not disclose the need to combine surfactants of N-acyl amino acid type, for instance disodium or monosodium cocoyl glutamate.

New technical solutions have recently been developed for preparing surfactant compositions which generate stable foams without the addition of foam-stabilizing additives. Mention may thus be made of the international patent application published under the publication number WO 2012/085391 A1, which discloses a process for drilling cavities in underground formations, using an aqueous fluid which is in the form of a foam obtained by mixing a gas or a gas mixture and an aqueous solution comprising an N-acyl derivative of glutamic acid, or a salt thereof, and/or of aspartic acid, or a salt thereof, for which the acyl radical includes from 8 to 18 carbon atoms. The foams generated by the aqueous solutions comprising such N-acyl derivatives of glutamic acid, or a salt thereof, and/or of aspartic acid, or a salt thereof, have the advantage both of being formed with a moderate expansion time and of showing mechanical properties that make them suitable for use in a step of rubble removal during an underground cavity drilling operation.

The American patent application published under the number US 2008/0233057A1 describes mild cleansing compositions comprising i) at least one surfactant of anionic, nonionic or amphoteric type, ii) at least one colorant, iii) at least one encapsulated colorant. The foaming surfactants included in this mild cleansing composition may be N-acyl amino acid derivatives, for instance sodium cocoyl glutamate, alkylpolyglycosides, for instance alkylpolyglycosides whose alkyl chain may include from 4 to 22 carbon atoms, more particularly from 8 to 16 carbon atoms.

The American patent application published under the number US 2004/0057922 A1 describes a cosmetic composition comprising, per 100% of its mass, i) from 2% to 50% by mass of at least one N-acyl amino acid derivative, and ii) from 50% to 98% by mass of at least one alkyl and/or alkenyl oligoglycoside. Said American patent application published under the number US 2004/0057922A1 also describes a comparative composition denoted (C3) comprising the sodium salt of cocoyl glutamate and C12-16 alkylpolyglucoside in a sodium cocoyl glutamate/ C12-16 alkylpolyglucoside mass ratio of 3/1. Said American patent application published under the number US 2004/0057922A1 discloses the characterization of the foaming properties of the compositions according to the invention under consideration and comparative composition (C3), thus showing that the foam generated by composition (C3) is unstable; the volume of foam measured 15 minutes after its formation being equal to zero.

The Japanese patent application published under the number JP2010-285397 describes compositions comprising at least one surfactant of N-acyl glutamic type, at least one surfactant of N-acyl aspartic type and at least one sugar-based surfactant selected from esters of sugar derivatives or alkylpolyglucosides. Among the surfactants of alkylpolyglucoside type which may be combined with the surfactant of N-acyl glutamic type and with the surfactant of N-acyl aspartic type, Japanese patent application JP2010-285397 specifically describes alkylpolyglucosides whose alkyl chain includes from 8 to 16 atoms, and more particularly from 8 to 12 carbon atoms (JP2010-285397, paragraph [0022]). Such compositions are described as having good foaming characteristics (JP2010-285397, paragraph [0048]) and of imparting substantial hair health and strength properties (JP2010-285397, paragraphs [0052], [0053], [0054]).

The Japanese patent application published under the number JP-H-04-114100 describes compositions comprising an N-acyl aspartic acid salt and at least one alkylpolyglucoside whose alkyl chain includes from 8 to 18 carbon atoms. Such compositions are used for preparing cleansing formulations with good detergent power, but also improved skin tolerance and imparting freshness sensory properties to the hair and the skin.

The international patent application published under the number WO2015/122371A1 describes cleansing compositions comprising at least one amphoteric surfactant, at least one nonionic surfactant and anionic surfactants, said anionic surfactants comprising at least one N-acyl glutamic acid derivative and at least one alkyl ether of a carboxylic acid and at least one alkylsulfosuccinic acid. These properties are described as generating a “rich” foam characterized by good sensory properties (WO2015/122371 A1, page 21, paragraph [0064]).

It has also been observed by the Applicant that the properties of the foams formed from aqueous solutions comprising N-acyl derivatives of glutamic acid or a salt thereof, and/or of aspartic acid or a salt thereof, required for the preparation of cleansing and foaming compositions for topical use, notably the expansion time and the foam stability, presented variations from one manufacturing batch to another, making them of random efficiency. These observed variations are associated with the variation of the mass proportion of residual fatty acids and/or with the mass proportion of inorganic salts present in the solutions of N-acyl derivatives of glutamic acid, or of a salt thereof, and/or of aspartic acid, or of a salt thereof, inherent in the implementation of their preparation processes, the essential steps of which and the essential operating parameters of which remain constant and unchanged.

There thus remains a need for an aqueous cleansing composition for topical use, which spreads easily on the skin without leaving a greasy or tacky residual film on the skin, which is capable of inducing the formation of a stable foam having a rapid expansion time.

This is why, according to a first aspect, one subject of the invention is a process for improving the foaming properties of a cleansing formulation for topical use (F₁), comprising, per 100% of its mass, from 0.5% to 10% by mass, more particularly from 1% to 10% by mass and even more particularly from 1% to 8% by mass of a composition (C₁), said composition (C₁) comprising, per 100% of its mass:

(α)—from 65% to 90% by mass, more particularly from 65% to 85% by mass and most particularly from 65% to 80% by mass of at least one compound of formula (I):

R₁—C(═O)—NH—CH(COOH)—(CH₂)₂—COOH (I)

in partially or totally salified acid form in which the group R₁—C(═O)— represents a linear or branched, saturated or unsaturated acyl radical including from 8 to 18 carbon atoms, and

(β)—from 10% to 35% by mass, more particularly from 15% to 35% by mass and most particularly from 20% to 35% by mass of at least one compound of formula (II):

R₁—C(═O)—OH (II)

in partially or totally salified acid form in which the group R₁ is as defined for formula (I), said process being characterized in that it comprises a step a1) of incorporating into said formulation (F₁) an effective amount of at least one composition (C₂) comprising, per 100% of its mass:

(γ)—from 14% to 80% by mass of a composition (C₃) or of a mixture of compositions (C₃), said composition (C₃) being represented by formula (III):

R₃—O-(G₃)ₚ-H (III)

in which R₃ represents a linear or branched, saturated or unsaturated aliphatic radical including from 12 to 16 carbon atoms, G₃ represents a reducing sugar residue and p represents a decimal number greater than or equal to 1.05 and less than or equal to 5, said composition (C₃) consisting of a mixture of compounds represented by formulae (III₁), (III₂), (III₃), (III₄) and (III₅):

R₃—O-(G₃)₁-H (III₁),

R₃—O-(G₃)₂-H (III₂),

R₃—O-(G₃)₃-H (III₃),

R₃—O-(G₃)₄-H (III₄),

R₃—O-(G₃)₅-H (III₅),

in the respective molar proportions a₁, a₂, a₃, a₄ and a₅ such that:
the sum a₁+a₂+a₃+a₄+a₅ is equal to 1, and
the sum a₁+2a₂+3a₃+4a₄+5a₅ is equal to p;

(δ)—from 0% to 3% by mass of at least one alcohol of formula (IV):

R₃—OH (IV)

in which R₃ is as defined for the preceding formula (III),

(ε)—from 20% to 80% of a composition (C₄) or of a mixture of compositions (C₄), said composition (C₄) being represented by formula (V):

R₄—O-(G₄)_{q}-H (V)

in which R₄ represents a linear aliphatic radical chosen from butyl, pentyl, hexyl and heptyl radicals, G₄ represents a reducing sugar residue and q represents a decimal number greater than or equal to 1.05 and less than or equal to 5, said composition (C₄) consisting of a mixture of compounds represented by formulae (V₁), (V₂), (V₃), (V₄) and (V₅):

R₄—O-(G₄)₁-H (V₁),

R₄—O-(G₄)₂-H (V₂),

R₄—O-(G₄)₃-H (V₃),

R₄—O-(G₄)₄-H (V₄),

R₄—O-(G₄)₅-H (V₅),

in the respective molar proportions a′₁, a′₂, a′₃, a′₄ and a′₅, such that:
the sum a′₁+a′₂+a′₃+a′₄+a′₅ is equal to 1, and
the sum a′₁+2a′₂+3a′₃+4a′₄+5a′₅ is equal to q; and

(η)—from 0% to 3% by mass of at least one alcohol of formula (VI):

R₄—OH (VI)

in which R₄ is as defined for the preceding formula (V).

In the definition of the process that is the subject of the invention, the term “effective amount of at least one composition (C₂)” means the amount of said composition (C₂) such that, once incorporated into the formulation (F1), it leads to a final formulation obtained via said process which:
generates a foam height of greater than or equal to 130 mm, 30 minutes after its formation under the operating conditions described in section 4 of the experimental part of the present specification;
shows a half-life time, determined according to the method described in section 4 of the experimental part of the present specification, of greater than or equal to 30 minutes;
shows an expansion time, determined according to the method described in section 4 of the experimental part of the present specification, of greater than or equal to 25 seconds;
shows a degree of expansion of greater than or equal to 4 and less than or equal to 20.

The degree of expansion (T_{F}) is the ratio between the volume of foam produced by a foaming composition to the volume of the foaming solution used. Consequently, the more the degree of expansion increases, the lighter the foam and the greater its volume, not allowing easy spreading on the skin.

In the context of the present invention, the term “compounds of formula (I) or (II) in partially or totally salified acid form” means that one, several or all of the carboxyl functions present in one or other of said compounds of formula (I) or (II) is either in acid form (—COOH) or in salified form (—COO⁻ M⁺). In the latter case, M⁺ represents a monovalent cation chosen from:
the ammonium cation,
monovalent cations of alkali metals, for example the sodium (Na⁺), potassium (K⁺) or lithium (Li⁺) cation,
(hydroxyalkyl)ammonium, bis(hydroxyalkyl)ammonium or tris(hydroxyalkyl)ammonium cations in which the hydroxyalkyl radical(s) include from 1 to 4 carbon atoms, for example 2-hydroxyethanammonium, 2-hydroxypropanammonium, bis(2-hydroxyethyl)ammonium and tris(2-hydroxyethyl)ammonium cations,
(alkyloxyalkyl)ammonium, bis(alkyloxyalkyl)ammonium or tris(alkyloxyalkyl)ammonium cations in which the alkyloxyalkyl radical(s) include from 2 to 6 carbon atoms, for example the 2-ethoxyethanammonium cation,
(hydroxyalkylaminoalkyl)ammonium, bis(hydroxyalkylaminoalkyl)ammonium or tris(hydroxyalkylaminoalkyl)ammonium cations in which the hydroxyalkylaminoalkyl radical(s) include from 2 to 6 carbon atoms, for example the 2-hydroxyethylaminomethanammonium cation and the 2-hydroxyethylaminoethanammonium cation.

The term “reducing sugar residue” denotes in the definition residues (G₃) and (G₄) of formulae (III) and (V) as defined previously, residues of saccharide derivatives without a glycoside bond established between an anomeric carbon and the oxygen of an acetal group, as defined in the reference publication: “Biochemistry, Daniel Voet/Judith G. Voet, page 250, John Wiley & Sons, 1990.”

The oligomeric structures (G₃)ₚ and (G₄)_{q} may be in any isomeric form, whether it is optical isomerism, geometrical isomerism or regioisomerism; it may also represent a mixture of isomers.

In formula (III) as defined above, the group R₃ is linked to G₃ via the anomeric carbon of the saccharide residue, so as to form an acetal function. Similarly, in formula (V) as defined above, the group R₄ is linked to G₄ via the anomeric carbon of the saccharide residue, so as to form an acetal function.

According to a particular aspect of the process as defined previously, in formula (III) and in formula (IV), respectively, G₃ and G₄, which may be identical or different, represent, independently of each other, a reducing sugar residue, glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose.

The term “linear or branched, saturated or unsaturated aliphatic radical including from 12 to 16 carbon atoms” notably denotes for R₃ in formulae (III) and (IV):
a linear alkyl radical chosen from dodecyl (n-C₁₂H₂₅—), tetradecyl (n-C₁₄H₂₉—) and hexadecyl (n-C₁₆H₃₂—) radicals,
a branched alkyl radical derived from the isoalkanols of formula (1):

(CH₃)(CH₃)CH—(CH₂)₁—CH₂—OH (1)

in which r represents an integer between 8 and 16, for example the isododecyl, isotridecyl, isotetradecyl, isopentadecyl or isohexadecyl radical;
a branched alkyl radical derived from a Guerbet alcohol of formula (2):

CH(CₛH₂ₛ₊₁)(CₜH₂ₜ₊₁)—CH₂—OH (2)

in which t is an integer between 2 and 12, s is an integer between 2 and 14 and the sum s+t is greater than or equal to 10 and less than or equal to 14, for example the 2-ethyldecyl, 2-butyloctyl, 2-ethyldodecyl, 2-butyldecyl, 2-hexyloctyl, 2-hexyldecyl or 2-butyldodecyl radical.

According to a particular aspect, said process for improving the foaming properties of a cleansing formulation for topical use (F₁) is characterized in that, in said composition (C₂), the proportions of alcohol of formula (VI) are zero.

According to another particular aspect of the process that is the subject of the present invention, the compounds of formula (I) and of formula (II) are partially or totally salified in sodium salt or potassium salt form.

According to another particular aspect, composition (C₁) as defined previously comprises, per 100% of its mass, from 65% to 90% by mass of one or more compounds of formula (I) and from 10% to 35% by mass of one or more compounds of formula (II); and more particularly from 65% to 85% by mass of one or more compounds of formula (I) and from 15% to 35% by mass of one or more compounds of formula (II).

In formulae (I) and (II) as defined previously, the radical R₁—(C═O)— more particularly represents an acyl radical chosen from octanoyl, decanoyl, ω-undecylenoyl, dodecanoyl, tetradecanoyl, hexadecanoyl, octadecanoyl, 9-octadecenoyl, 9,12-octadecadienoyl and 9,12,15-octadecatrienoyl radicals.

The compounds of formula (I) as described previously are generally obtained by N-acylation of the corresponding amino acids or of salts thereof. It is described, for example, in the international patent application published under the number WO 98/09611. It is performed equivalently on an amino acid or on an amino acid mixture. The acylating agent generally consists of an activated derivative of the carboxylic acid of formula:

R₁—C(═O)—OH,

in which R₁ is as defined previously, such as a symmetrical anhydride of this acid, the methyl ester of this acid, or an acid halide such as the acid chloride or the acid bromide. It may also consist of a mixture of activated derivatives of carboxylic acids obtained from natural oils or fats of animal or plant origin such as coconut kernel oil, coconut oil, palm kernel oil, palm oil, soybean oil, rapeseed oil, corn oil, beef tallow, spermaceti oil or herring oil.

According to another particular aspect, a subject of the invention is said process as defined previously, characterized in that said composition (C₁) is obtained via a process comprising at least:
one step A) of acylation of a compound of formula (VII):

NH₂—CH(COOH)—(CH₂)₂—COOH (VII),

in partially or totally salified acid form, with a mixture of acid chlorides comprising, per 100 mol %, from 40 mol % to 60 mol % of dodecanoyl chloride, from 10 mol % to 20% of tetradecanoyl chloride, from 5 mol % to 15 mol % of decanoyl chloride and from 5 mol % to 15 mol % of octanoyl chloride, and optionally and up to a maximum of 100 mol %, of hexadecanoyl chloride and/or of octadecanoyl chloride and/or of 9-octadecenoyl chloride and/or of octadeca-9,12-dienoyl chloride.

According to a more particular aspect, the mixture of acid chlorides used comprises, per 100 mol %, 11 mol % of octanoyl chloride, 9.5 mol % of decanoyl chloride, 51 mol % of dodecanoyl chloride, 15.5 mol % of tetradecanoyl chloride, 6.5 mol % of hexadecanoyl chloride, 2 mol % of octadecanoyl chloride, 3 mol % of 9-octadecenoyl chloride and 1.5 mol % of octadeca-9,12-dienoyl chloride.

According to another particular aspect, a subject of the invention is said process as defined previously, characterized in that the compound(s) of formula (I) are chosen from monosodium N-cocoyl glutamate, monopotassium N-cocoyl glutamate, disodium N-cocoyl glutamate and dipotassium N-cocoyl glutamate.

According to another particular aspect, a subject of the invention is the process as defined previously, characterized in that said composition (C₂) comprises, per 100% of its mass:

(γ)—a mass proportion of said composition (C₃) of greater than or equal to 14% and less than 70%, and

(δ)—a mass proportion of said alcohol of formula (IV) of greater than or equal to 0% and less than or equal to 3%,

(ε)—a mass proportion of said composition (C₄) of greater than or equal to 30% and less than or equal to 80%, and

(η)—a mass proportion of said alcohol of formula (VI) of greater than or equal to 0% and less than or equal to 3%.

According to an even more particular aspect, a subject of the invention is said process as defined previously, characterized in that said composition (C₂) as defined previously comprises, per 100% of its mass, a mass proportion of said composition (C₃) of greater than or equal to 14% and less than or equal to 65%, a mass proportion of said alcohol of formula (IV) of greater than or equal to 0% and less than or equal to 3%, a mass proportion of said composition (C₄) of greater than or equal to 35% and less than or equal to 80% and a mass proportion of said alcohol of formula (VI) of greater than or equal to 0% and less than or equal to 3%.

According to another particular aspect, a subject of the invention is the process as defined previously, characterized in that, in said formula (III), G₃ represents a reducing sugar residue chosen from glucose, xylose and arabinose residues.

According to another particular aspect, a subject of the invention is the process as defined previously, characterized in that, in said formula (III), p represents a decimal number greater than or equal to 1.05 and less than or equal to 2.5, more particularly greater than or equal to 1.05 and less than or equal to 2.0 and even more particularly greater than or equal to 1.25 and less than or equal to 2.0.

According to another particular aspect, a subject of the invention is the process as defined previously, characterized in that, in formulae (III) and (IV), the radical R₃ represents a linear alkyl radical chosen from dodecyl (n-C₁₂H₂₅—), tetradecyl (n-C₁₄H₂₉—) and n-hexadecyl (n-C₁₆H₃₂—) radicals.

According to another particular aspect, a subject of the invention is the process as defined previously, characterized in that, in formula (V), G₄ represents a reducing sugar residue chosen from glucose, xylose and arabinose residues.

According to another particular aspect, a subject of the invention is the process as defined previously, characterized in that, in formula (V), q represents a decimal number greater than or equal to 1.05 and less than or equal to 2.5, more particularly greater than or equal to 1.05 and less than or equal to 2.0 and even more particularly greater than or equal to 1.25 and less than or equal to 2.0.

According to a particular aspect, a subject of the invention is the process as defined previously, characterized in that, in formulae (V) and (VI), R₄ represents a linear alkyl radical chosen from hexyl (n-C₆H₁₃—) and n-heptyl (n-C₇H₁₅—) radicals.

According to an even more particular aspect, in formulae (V) and (VI), R₄ represents the heptyl radical (n-C₇H₁₅—).

According to another even more particular aspect, in formulae (V) and (VI), R₄ represents the n-hexyl radical (n-C₆H₁₃—).

According to another particular aspect, a subject of the invention is the process as defined previously, characterized in that said composition (C₂) comprises a mixture of compositions (C₃) and of compositions (C₄), said mixture comprising, per 100% of its mass:
(γ₁)—from 13.6% to 44.4% by mass, more particularly from 17% to 44.4% by mass, of a composition (C₃) represented by formula (III) in which R₃ represents the dodecyl radical (n-C₁₂H₂₅—),
(γ₂)—from 5% to 16.25% by mass, more particularly from 6.25% to 16.25% by mass, of a composition (C₃) represented by formula (III) in which R₃ represents the tetradecyl radical (n-C₁₄H₁₉—), and
(γ₃)—from 1.4% to 4.55% by mass, more particularly from 1.75% to 4.55% by mass, of a composition (C₃) represented by formula (III) in which R₃ represents the hexadecyl radical (n-C₁₆H₃₂—), and
(ε₁)—from 35% to 80% by mass, more particularly from 35% to 75% by mass, of a composition (C₄) represented by formula (V) in which R₄ represents the heptyl radical (n-C₇H₁₅—).

According to another particular aspect, a subject of the invention is the process as defined previously, characterized in that said composition (C₂) comprises a mixture of compositions (C₃) and of compositions (C₄), said mixture comprising, per 100% of its mass:
from 13.6% to 44.4% by mass, more particularly from 17% to 44.4% by mass, of a composition (C₃) represented by formula (III) in which R₃ represents the dodecyl radical (n-C₁₂H₂₅—),
from 5% to 16.25% by mass, more particularly from 6.25% to 16.25% by mass, of a composition (C₃) represented by formula (III) in which R₃ represents the tetradecyl radical (n-C₁₄H₂₉—), and
from 1.4% to 4.55% by mass, more particularly from 1.75% to 4.55% by mass, of a composition (C₃) represented by formula (III) in which R₃ represents the hexadecyl radical (n-C₁₆H₃₂—), and
from 35% to 80% by mass, more particularly from 35% to 75% by mass, of a composition (C₄) represented by formula (V) in which R₄ represents the 2-ethylhexyl radical.

According to another particular aspect, a subject of the invention is the process as defined previously, characterized in that composition (C₂) comprises a mixture of compositions (C₃) comprising, per 100% of its mass:
from 13.6% to 44.4% by mass, more particularly from 17% to 44.4% by mass, of a composition (C₃) represented by formula (III) in which R₃ represents the dodecyl radical (n-C₁₂H₂₅—),
from 5% to 16.25% by mass, more particularly from 6.25% to 16.25% by mass, of a composition (C₃) represented by formula (III) in which R₃ represents the tetradecyl radical (n-C₁₄H₂₉—), and
from 1.4% to 4.55% by mass, more particularly from 1.75% to 4.55% by mass, of a composition (C₃) represented by formula (III) in which R₃ represents the hexadecyl radical (n-C₁₆H₃₂—), and
from 35% to 80% by mass, more particularly from 35% to 75% by mass, of a composition (C₄) represented by formula (V) in which R₄ represents the hexadecyl radical (n-C₁₆H₃₂—).

According to a more particular aspect, the process as defined previously is characterized in that, in formula (III), G₃ represents a reducing sugar residue chosen from glucose, xylose and arabinose residues, p represents a decimal number greater than or equal to 1.05 and less than or equal to 2.5, more particularly greater than or equal to 1.05 and less than or equal to 2.0 and even more particularly greater than or equal to 1.25 and less than or equal to 2.0, and R₃ represents a linear alkyl radical chosen from dodecyl (n-C₁₂H₂₅), tetradecyl (n-C₁₄H₂₉—) and hexadecyl (n-C₁₆H₃₂) radicals.

According to an even more particular aspect, the process as defined previously is characterized in that, in formula (III), G₃ represents a glucose residue, p represents a decimal number greater than or equal to 1.05 and less than or equal to 2.5, and R₃ represents a linear alkyl radical chosen from dodecyl (n-C₁₂H₂₅), tetradecyl (n-C₁₄H₂₉—) and hexadecyl (n-C₁₆H₃₂) radicals.

According to another more particular aspect, the process as defined previously is characterized in that, in formula (III), G₃ represents a xylose residue, p represents a decimal number greater than or equal to 1.05 and less than or equal to 2.5, and R₃ represents a linear alkyl radical chosen from the dodecyl (n-C₁₂H₂₅—) radical, the tetradecyl (n-C₁₄H₂₉—) radical and the hexadecyl (n-C₁₆H₃₂—) radical.

According to another more particular aspect, the process as defined previously is characterized in that, in formula (V), G₄ represents a reducing sugar residue chosen from glucose, xylose and arabinose residues, q represents a decimal number greater than or equal to 1.05 and less than or equal to 2.5, more particularly greater than or equal to 1.05 and less than or equal to 2.0 and even more particularly greater than or equal to 1.25 and less than or equal to 2.0, and R₄ represents an aliphatic alkyl radical chosen from hexyl (n-C₆H₁₃—), heptyl (n-C₇H₁₅—) and 2-ethylhexyl radicals.

According to an even more particular aspect, the process as defined previously is characterized in that, in formula (V), G₄ represents a glucose residue, q represents a decimal number greater than or equal to 1.05 and less than or equal to 2.5, and R₄ represents an aliphatic alkyl radical chosen from hexyl (n-C₆H₁₃—), heptyl (n-C₇H₁₅—) and 2-ethylhexyl radicals.

According to an even more particular aspect, the process as defined previously is characterized in that, in formula (V), G₄ represents a glucose residue, q represents a decimal number greater than or equal to 1.05 and less than or equal to 2.5, and R₄ represents the heptyl (n-C₇H₁₅—) radical.

According to another even more particular aspect, the process as defined previously is characterized in that, in formula (V), G₄ represents the xylose residue, q represents a decimal number greater than or equal to 1.05 and less than or equal to 2.5, and R₄ represents an aliphatic alkyl radical chosen from hexyl (n-C₆H₁₃—), heptyl (n-C₇H₁₅—) and 2-ethylhexyl radicals.

According to a most particular aspect, the process as defined previously is characterized in that, in formula (V), G₄ represents the xylose residue, q represents a decimal number greater than or equal to 1.05 and less than or equal to 2.5, and R₄ represents the heptyl (n-C₇H₁₅—) radical.

According to an even more particular aspect, the process as defined previously is characterized in that composition (C₃) is obtained by performing a process comprising at least one step A′) of glycosylation of:
1 to 5 molar equivalents, more particularly from 2 to 4 molar equivalents, even more particularly from 3 to 4 molar equivalents, of a mixture of alcohols comprising, per 100 mol %:
from 40 mol % to 90 mol % of 1-dodecanol, more particularly from 65 mol % to 90 mol % and even more particularly from 75 mol % to 90 mol %,
from 9 mol % to 40 mol % of 1-tetradecanol, more particularly from 9 mol % to 20 mol % and even more particularly from 9 mol % to 20 mol %, and
from 1 mol % to 20 mol % of 1-hexadecanol, more particularly from 1 mol % to 15% and even more particularly from 1 mol % to 5 mol %,
with 1 molar equivalent of a reducing sugar of formula (VII):

HO-(G₃)-H (VII)

in which G₃ represents a reducing sugar residue chosen from glucose, xylose and arabinose residues.

According to another particular aspect, the process as defined previously is characterized in that composition (C₄) is obtained by performing a process comprising at least one step A₁′ of glycosylation of:
1 to 4 molar equivalents, more particularly from 1 to 3 molar equivalents, even more particularly from 2 to 3 molar equivalents of at least one alcohol of formula (VI) with 1 molar equivalent of a reducing sugar of formula (VIII):

HO-(G₄)-H (VIII)

in which G₄ represents a reducing sugar residue chosen from glucose, xylose and arabinose residues.

According to another particular aspect, the process as defined previously is characterized in that composition (C₃) is obtained by performing a process comprising at least one step A′ of glycosylation generally performed with mechanical stirring, by placing 1 molar equivalent of a reducing sugar (G₃) in contact with from 1 to 5 molar equivalents of the mixture of 1-dodecanol, 1-tetradecanol and 1-hexadecanol as described previously, in the presence of an acidic catalytic system, under predetermined temperature and partial vacuum conditions.

Similarly, step A₁′ of the process for preparing composition (C₄), as defined previously, is generally performed with mechanical stirring, by placing 1 molar equivalent of a reducing sugar (G₄) in contact with from 1 to 4 molar equivalents of at least one alcohol of formula (V), in the presence of an acidic catalytic system, under predetermined temperature and partial vacuum conditions.

Such temperature and partial vacuum conditions are, for example, temperature values of between 70° C. and 130° C. and a partial vacuum of between 300 mbar (3×10⁴ Pa) and 20 mbar (2×10³ Pa). The implementation of step A′ and of step A₁′ of glycosylation makes it possible to form, respectively, composition (C₃), i.e. a mixture of compounds represented by the formulae (III₁), (III₂), (III₃), (III₄) and (III₅) as defined previously, and optionally of an excess of the alcohol of formula (IV) or of the mixture of alcohols of formula (IV), and composition (C₄), i.e. a mixture of compounds represented by the formulae (V₁), (V₂), (V₃), (V₄) and (V₅) as defined previously, and optionally of an excess of the alcohol of formula (VI).

If necessary or if desired, step A′ or step A₁′ of the process for preparing the respective compositions (C₃) and (C₄) as defined previously may be followed, respectively, by a step B′ or a step B₁′ of removal of the alcohols, respectively, of formula (IV), or of the mixture of alcohols of formula (IV), and of formula (VI), which have not reacted during step A′) or step A₁′.

Such a preparation process may be completed, if necessary or if desired, by neutralization, filtration and decolorization operations.

The term “acidic catalytic system” denotes, in step A′ and in step A₁′ of the process defined above, strong acids such as sulfuric acid, hydrochloric acid, phosphoric acid, nitric acid, methanesulfonic acid, para-toluenesulfonic acid, trifluoromethanesulfonic acid, hypophosphorous acid, hyponitrous acid, polyphosphoric acid, or ion-exchange resins.

During step B′) or step B′1) of the process as described above, the alcohols, respectively, of formula (IV), or the mixture of alcohols of formula (IV) or the alcohol of formula (V) are removed according to methods known to those skilled in the art, for instance by distillation, such as thin-film distillation, molecular distillation or solvent extraction.

According to a particular aspect, the subject of the invention is the process as defined previously, which is characterized in that the mass ratio

Δ=Mass of compound(s) of formula (I)/[Mass of composition (C₃)+Mass of composition (C₄)], is greater than or equal to 20/80 and less than or equal to 65/35, more particularly greater than or equal to 25/75 and less than or equal to 65/35.

According to another particular aspect, the subject of the invention is the process as defined previously, which is characterized in that the mass ratio

Δ₁=Mass of composition (C₃)/Mass of composition (C₄), is greater than or equal to 20/80 and less than or equal to 70/30, more particularly greater than or equal to 20/80 and less than or equal to 65/35, even more particularly greater than or equal to 25/75 and less than or equal to 65/35.

A subject of the invention is also a formulation (F₂) for topical use, characterized in that it comprises, per 100% by mass:
a)—from 0.5% to 10% by mass, more particularly from 1% by mass to 10% by mass and even more particularly from 1% by mass to 8% by mass of a composition (C₁), said composition (C₁) comprising, per 100% of its mass:
   (α)—from 65% to 90% by mass, more particularly from 65% by mass to 85% by mass and most particularly from 65% by mass to 80% by mass of at least one compound of formula (I):

R₁—C(═O)—NH—CH(COOH)—(CH₂)₂—COOH (I)
in partially or totally salified acid form, in which the group R₁—C(═O)— represents a linear or branched, saturated or unsaturated acyl radical including from 8 to 18 carbon atoms, and
(β)—from 10% to 35% by mass, more particularly from 15% by mass to 35% by mass and most particularly from 20% by mass to 35% by mass of at least one compound of formula (II):

R₁—C(═O)—OH (II)
in partially or totally salified acid form, in which the group R₁ is as defined for formula (I),
b)—from 0.1% to 10% by mass, more particularly from 0.1% by mass to 5% by mass and even more particularly from 0.5% by mass to 5% by mass of at least one composition (C₂) as defined in any one of claims 1 to 12, and
c)—from 3.4% to 20% by mass, more particularly from 3.9% by mass to 20% by mass and even more particularly from 4% by mass to 17% by mass of at least one foaming and/or detergent surfactant chosen from anionic and amphoteric foaming and/or detergent surfactants, and
d)—from 60% to 96% by mass, more particularly from 65% by mass to 95% by mass and even more particularly from 70% by mass to 94.5% by mass of water.

The expression “for topical use” used in the definition of the formulation as defined above means that said cleansing formulation is used by application to the skin, the hair, the scalp or the mucous membranes, whether it is a direct application in the case of a cosmetic, dermocosmetic, dermopharmaceutical or pharmaceutical cleansing formulation or an indirect application, for example in the case of a body hygiene formulation in the form of a textile or paper wipe, or sanitary products intended to be in contact with the skin or the mucous membranes.

The term “foaming and/or detergent surfactants” denotes, in the definition of the formulation (F₂) for topical use as defined above, compounds of amphiphilic structure which are used to generate the foam in aqueous solution and/or to allow the cleansing of soiling from human skin, and more particularly anionic and/or amphoteric foaming and/or detergent surfactants.

As examples of foaming and/or detergent anionic surfactants that may be present in formulation (F₂) for topical use that is the subject of the present invention, mention may be made of alkali metal salts, alkaline-earth metal salts, ammonium salts, amine salts, salts of amino alcohols, of alkyl ether sulfates, of alkylamido ether sulfates, of alkylarylpolyether sulfates, of α-olefinsulfonates, of paraffin sulfonates, of alkyl phosphates, of alkyl ether phosphates, of alkylsulfonates, of alkylamidesulfonates, of alkylarylsulfonates, of alkylcarboxylates, of alkylsulfosuccinates, of alkyl ether sulfosuccinates, of alkylamidesulfosuccinates, of alkylsarcosinates, of acylisethionates, of N-acyltaurates or of acyllactylates.

According to a particular aspect, the foaming and/or detergent amphoteric surfactant present in formulation (F₂) for topical use that is the subject of the present invention is a compound represented by formula (VII):

[R′₁—O—SO₃⁻]ₜ, X^{t+} (VII)

in which:
R′₁ represents a linear or branched, saturated or unsaturated aliphatic hydrocarbon-based radical including from 6 to 22 carbon atoms,
t represents an integer equal to 1, 2 or 3,
X^{t+} represents a cation of valency t,
   or a mixture of compounds of formula (VII).

According to a particular aspect, the subject of the invention is a formulation (F₂) for topical use as defined previously, for which, in the definition of compound (VII), the radical R′₁ represents a radical chosen from n-octyl, n-decyl, n-dodecyl, n-tetradecyl, n-hexadecyl and n-octadecyl radicals.

According to another particular aspect, the subject of the invention is a formulation (F₂) for topical use as defined previously, for which, in the definition of compound (VII), X^{t+}represents a cation chosen from:
i) when t is equal to 1, the cations of an alkali metal, the ammonium ion, the ammonium ion of an N-alkylamine, the ammonium ion of an N-(hydroxyalkyl)amine,
ii) when t is equal to 2, the cations of an alkaline-earth metal, and
iii) when t is equal to 3, the cations of aluminum or boron.

According to another more particular aspect, the subject of the invention is a formulation (F₂) for topical use as defined previously, for which, in the definition of compound (VII), the cation X^{t+} represents a cation selected from sodium, potassium, ammonium, N-ethylammonium, N,N-diethylammonium, N,N,N-triethylammonium and N-(2-hydroxyethyl)ammonium cations, and the N,N-bis(2-hydroxyethyl)ammonium or N,N,N-tris(2-hydroxyethyl)ammonium ion.

According to a more particular aspect, the subject of the invention is a formulation (F₂) for topical use as defined previously, for which, in the definition of compound (VII):
the radical R′₁ represents a radical chosen from n-octyl, n-decyl, n-dodecyl, n-tetradecyl and n-hexadecyl radicals, and
X^{t+} represents a cation selected from cations of an alkali metal, of an alkaline-earth metal, ammonium, N-ethylammonium, N,N-diethylammonium, N,N,N-triethylammonium, N-(2-hydroxyethyl)ammonium, and the N,N-bis(2-hydroxyethyl)ammonium or N,N,N-tris(2-hydroxyethyl)ammonium ion.

According to another particular aspect, the foaming and/or detergent anionic surfactant present in formulation (F₂) for topical use that is the subject of the present invention is a compound of formula (VIII):

[R′₂—O—(CH₂—CH₂—O)ₛSO₃]ᵣY (VIII)

in which:

R₂ represents a linear or branched, saturated or unsaturated aliphatic hydrocarbon-based radical including from 6 to 22 carbon atoms,

s represents a decimal number between 1 and 10 and preferably between 2 and 4,

r is an integer equal to 1 or 2 and

Y represents the cation of an alkali metal or of an alkaline-earth metal, the ammonium ion, the hydroxyethylammonium ion or the tris(hydroxyethyl)ammonium ion.

In formula (VIII) as defined above, Y represents, for example, sodium, magnesium or the ammonium ion.

According to a more particular aspect, the subject of the invention is a formulation (F₂) for topical use as defined previously, for which, in the definition of compound (VIII):
the radical R′₂ represents a radical chosen from n-octyl, n-decyl, n-dodecyl, n-tetradecyl and n-hexadecyl radicals, and
Y represents a cation selected from cations of an alkali metal, of an alkaline-earth metal, ammonium, N-ethylammonium, N,N-diethylammonium, N,N,N-triethylammonium, N-(2-hydroxyethyl)ammonium, and the N,N-bis(2-hydroxyethyl)ammonium or N,N,N-tris(2-hydroxyethyl)ammonium ion.

As examples of foaming and/or detergent amphoteric surfactants that may be present in formulation (F₂) for topical use which is a subject of the present invention, mention may be made particularly of alkylbetaines, alkylamidobetaines, sultaines, alkylamidoalkylsulfobetaines, imidazoline derivatives, phosphobetaines, amphopolyacetates and amphopropionates.

According to a particular aspect, the foaming and/or detergent amphoteric surfactant present in formulation (F₂) for topical use that is the subject of the present invention is a compound of formula (IX):

R′₃—C(O)—NH(CH₂)_{q}—N⁺(R′₄)(R′₅)—(CH₂)ᵥ—CO₂⁻ (IX)

in which R′₃ represents a saturated or unsaturated, linear or branched aliphatic hydrocarbon-based radical including from 7 to 21 carbon atoms, R′₄ and R′₅ represent, independently of each other, a saturated or unsaturated, linear or branched aliphatic radical, optionally substituted with a hydroxyl group, including from 1 to 4 carbon atoms, q represents an integer between 2 and 6 and v represents an integer equal to 1 or 2, or a mixture of compounds of formula (IX).

In formula (IX) as defined above, R′₃—C(O)— represents, for example, the octanoyl radical, the decanoyl radical, the lauroyl radical or the cocoyl radical.

In formula (IX) as defined above, q is, for example, equal to 3.

In formula (IX) as defined above, R′₄ and R′₅ represent a methyl radical.

According to a more particular aspect of the present invention, a subject thereof is a formulation (F₂) for topical use, for which the foaming and/or detergent amphoteric surfactant of formula (IX) is cocamidopropyl betaine.

Formulation (F₂) for topical use, which is a subject of the present invention, may be packaged in pressurized form in an aerosol device or in a device of “pump-bottle” type, in a device equipped with a perforated wall, for example a grille, or in a device equipped with a ball applicator (known as a “roll-on”). When it is packaged in bottles, formulation (F₂) for topical use according to the invention as defined previously may be applied in the form of fine droplets by means of mechanical pressurization devices or via propellant gas devices. Among the propellants that may be combined with formulation (F₂) for topical use according to the invention are hydrofluoro compounds, for instance dichlorodifluoromethane, trichlorofluoromethane, difluoroethane, isobutane, butane and propane.

Formulation (F₂) for topical use as defined previously may also include excipients and/or active principles commonly used in the field of formulations for topical use, in particular cosmetic, dermocosmetic, pharmaceutical or dermopharmaceutical formulations.

Formulation (F₂) for topical use, which is the subject of the present invention and as defined previously, may also comprise one or more auxiliary compounds chosen from thickening and/or gelling surfactants, thickeners and/or gelling agents, stabilizers, film-forming compounds, solvents and cosolvents, hydrotropic agents, plasticizers, opacifiers, nacreous agents, sequestrants, chelating agents, antioxidants, fragrances, essential oils, preserving agents, conditioning agents, deodorants, bleaching agents intended for bleaching bodily hair and the skin, active agents intended to provide a treating and/or protective action to the skin or the hair, sunscreens, mineral fillers or pigments, particles that give a visual effect or that are intended for encapsulating active agents, exfoliant particles, texture agents, optical brighteners and insect repellents.

Among the water-soluble antioxidants that may be combined with formulation (F₂) for topical use according to the invention are ascorbic acid, glutathione, tartaric acid, oxalic acid and tetrasodium glutamate diacetate.

Among the water-soluble sequestrants that may be combined with formulation (F₂) for topical use according to the invention are ethylenediaminetetraacetic acid (EDTA) salts, for instance the sodium salt of EDTA, diethylenetriaminepentaacetic acid (DTPA) salts, for instance the sodium salts of DTPA, and acetyl glutamic acid (Dissolvine range).

Among the water-soluble dyes that may be combined with formulation (F₂) for topical use according to the invention are caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochineal carmine (CI 15850, CI 75470), Ext. Violet 2, Red 6-7, Ferric Ferrocyanide, Ultramarines, Acid Yellow 3/Yellow 10, Acid Blue 3 and Yellow 10.

Among the color-stabilizing water-soluble agents that may be combined with formulation (F₂) for topical use according to the invention are tris(tetramethylhydroxypiperidinol) citrate, sodium benzotriazolyl butylphenol sulfonate and benzotriazolyl dodecyl p-cresol.

As examples of texture agents optionally present in formulation (F₂) for topical use which is a subject of the present invention, mention may be made of N-acylamino acid derivatives, for example lauroyl lysine sold under the name Aminohope™LL, octenyl starch succinate sold under the name DryfloTM, myristyl polyglucoside sold under the name Montanov™ 14, cellulose fibers, cotton fibers, chitosan fibers, talc, sericite, mica and perlite.

Examples of active principles optionally present in formulation (F₂) for topical use that is a subject of the present invention include:
vitamins and derivatives thereof, for example retinol (vitamin A) and esters thereof (for example retinyl palmitate), ascorbic acid (vitamin C) in salt form and esters thereof, sugar derivatives of ascorbic acid (for example ascorbyl glucoside), tocopherol (vitamin E) and esters thereof (for example tocopheryl acetate), vitamin B3 or B10 (niacinamide and derivatives thereof); compounds having a lightening or depigmenting action on the skin, for example Sepiwhite™ MSH, arbutin, kojic acid, hydroquinone, Vegewhite™, Gatuline™ Synerlight™, Biowhite™, Phytolight™, Dermalight™, Clariskin™, Melaslow™, Dermawhite™, Ethioline, Melarest™, Gigawhite™, Albatine™ and Lumiskin™;
compounds with calmative action, such as Sepicalm™ S, allantoin and bisabolol;
anti-inflammatory agents;
compounds with moisturizing action, for example diglycerol, triglycerol, urea, hydroxyureas, glycerol glucoside, diglycerol glucoside, polyglyceryl glucosides, erythrityl glucoside, sorbityl glucoside, xylityl glucoside, the composition sold under the brand name Aquaxyl™ comprising xylityl glucoside, anhydroxylitol and xylitol;
compounds with slimming or lipolytic action, such as caffeine or derivatives thereof, Adiposlim™ and Adipoless™;
plant extracts rich in tannins, polyphenols and/or isoflavones, for example grape extracts, pine extracts, wine extracts, olive extracts; soybean extracts, for example Raffermine™; wheat extracts, for example Tensine™ or Gliadine™; terpene-rich plant extracts; freshwater or seawater algal extracts; marine extracts in general such as corals;
compounds with antimicrobial action or with purifying action, for example Lipacide™ C8BG, Lipacide™ UG, Sepicontrol™ A5; Octopirox™ or Sensiva™ SC50;
compounds with an energizing or stimulating property, such as Physiogenyl™, panthenol and derivatives thereof such as Sepicap™ MP;
antiaging active agents such as Sepilift™ DPHP, Lipacide™ PVB, Sepivinol™ Sepivital™ Manoliva™, Phyto-Age™, Timecode™ or Survicode™;
active agents for combating photoaging;
active agents for increasing the synthesis of extracellular matrix components, for example collagen, elastins and glycosaminoglycans;
active agents acting favorably on chemical cellular communication, such as cytokines, or physical cellular communication, such as integrins;
active agents which create a “heating” sensation on the skin, such as skin capillary circulation activators (for example nicotinic acid derivatives) or products which create a “freshness” sensation on the skin (for example menthol and derivatives thereof);
active agents which improve the skin capillary circulation, for example venotonic agents; draining active agents; decongesting active agents, for example extracts of Ginkgo biloba, ivy, common horse chestnut, bamboo, ruscus, butcher's-broom, Centella asiatica, fucus, rosemary or willow;
active agents acting as skin-tautening agents, for example plant protein hydrolyzates, hydrolyzates of marine origin, for instance hydrolyzates of laminaria extracts, fish cartilage hydrolyzates, marine elastin, the product sold by the company SEPPIC under the brand name Sesaflash™, and collagen solutions;
skin tanning or browning agents, for example dihydroxyacetone, isatin, alloxan or ninhydrin, glyceraldehyde, mesotartaric aldehyde, glutaraldehyde or erythrulose.

As examples of deodorants optionally present in formulation (F₂) for topical use which is a subject of the present invention, mention may be made of alkali metal silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

As examples of thickeners or gelling agents that may be present in formulation (F₂) for topical use that is a subject of the present invention, mention may be made of linear or branched or crosslinked polymers of polyelectrolyte type, such as the partially or totally salified acrylic acid homopolymer, the partially or totally salified methacrylic acid homopolymer, the partially or totally salified 2-methyl-[(1-oxo-2-propenyl)amino]-1-propanesulfonic acid (AMPS) homopolymer, copolymers of acrylic acid and of AMPS, copolymers of acrylamide and of AMPS, copolymers of vinylpyrrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or methacrylic acid and of alkyl acrylates, the carbon chain of which comprises between 4 and 30 carbon atoms and more particularly between 10 and 30 carbon atoms, copolymers of AMPS and of alkyl acrylates, the carbon chain of which comprises between 4 and 30 carbon atoms and more particularly between 10 and 30 carbon atoms, linear, branched or crosslinked terpolymers of at least one monomer having a free, partially salified or totally salified strong acid function, with at least one neutral monomer, and at least one monomer of formula (VIII):

CH₂═C(R′₃)—C(═O)—[CH₂—CH₂—O]ₙ—R′₄ (VIII)

in which R^{′}₃ represents a hydrogen atom or a methyl radical, R^{′}₄ represents a linear or branched alkyl radical including from 8 to 30 carbon atoms and n represents a number greater than or equal to 1 and less than or equal to 50.

The linear or branched or crosslinked polymers of polyelectrolyte type that may be combined with formulation (F₂) for topical use that is a subject of the present invention may be present in the form of a solution, an aqueous suspension, a water-in-oil emulsion, an oil-in-water emulsion, a powder, for example the products sold under the names Simulgel™ EG, Simulgel™EPG, Sepigel™ 305, Simulgel™ 600, Simulgel™ NS, Simulgel™ INS 100, Simulgel™ FL, Simulgel™ A, Simulgel™ SMS 88, Sepinov™EMT 10, Sepiplus™400, Sepiplus™265, Sepiplus™S, Sepimax™Zen, Aristoflex™AVC, Aristoflex™AVS, Novemer™EC-1, Novemer™EC 2, Aristoflex™HMB, Cosmedia™SP, Flocare™ET 25, Flocare™ET 75, Flocare™ET 26, Flocare™ET 30, Flocare™ET 58, Flocare™PSD 30, viscolam™AT 64 and Viscolam™AT 100; polysaccharides constituted solely of saccharides, such as glucans or glucose homopolymers, glucomannoglucans, xyloglucans, galactomannans in which the degree of substitution (DS) of the D-galactose units on the D-mannose main chain is between 0 and 1 and more particularly between 1 and 0.25, for instance galactomannans originating from cassia gum (DS=⅕), from locust bean gum (DS=¼), from tara gum (DS=⅓), from guar gum (DS=½) or from fenugreek gum (DS=1); polysaccharides constituted of saccharide derivatives, such as galactan sulfates and more preferentially carrageenans and agar, uronans and more particularly algins, alginates and pectins, heteropolymers of saccharides and of uronic acids and more particularly xanthan gum, gellan gum, gum arabic extrudates and karaya gum extrudates, glucosaminoglycans; cellulose, cellulose derivatives such as methyl-cellulose, ethyl-cellulose, hydroxypropyl cellulose, silicates, starch, hydrophilic starch derivatives, polyurethanes.

As examples of oils that may be present in formulation (F₂) for topical use that is a subject of the present invention, mention may be made of mineral oils such as liquid paraffin, liquid petroleum jelly, isoparaffins or white mineral oils; oils of animal origin such as squalene or squalane; plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cotton oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, coriander seed oil, beechnut oil, beauty-leaf oil, sisymbrium oil, avocado oil, calendula oil, oils derived from flowers or vegetables, ethoxylated plant oils; synthetic oils, for instance fatty acid esters such as butyl myristate, propyl myristate, isopropyl myristate, cetyl myristate, isopropyl palmitate, octyl palmitate, butyl stearate, hexyldecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, for instance glyceryl triheptanoate, alkylbenzoates, hydrogenated oils, poly(α-olefins), polyolefins such as poly(isobutane), synthetic isoalkanes, for instance isohexadecane, isododecane, perfluorinated oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups. In the present patent application, the term “oils” refers to compounds and/or mixtures of compounds which are water-insoluble, and which have a liquid appearance at a temperature of 25° C.

As examples of waxes that may be present in formulation (F₂) for topical use that is a subject of the present invention, mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature. In the present patent application, the term “waxes” refers to compounds and/or mixtures of compounds which are water-insoluble, and which have a solid appearance at a temperature of greater than or equal to 45° C.

As examples of emulsifying nonionic surfactants that may be combined with formulation (F₂) for topical use that is a subject of the present invention, mention may be made of fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters, ethoxylated mannitan esters; sucrose esters; methyl glucose esters; alkyl polyglycosides including a linear or branched, saturated or unsaturated aliphatic radical, and including from 14 to 36 carbon atoms, for instance tetradecyl polyglucoside, hexaldecyl polyglucoside, octadecyl polyglucoside, hexadecyl polyxyloside, octadecyl polyxyloside, eicosyl polyglucoside, dodecosyl polyglucoside, (2-octyldodecyl) polyxyloside, (12-hydroxystearyl) polyglucoside; compositions of linear or branched, saturated or unsaturated fatty alcohols including from 14 to 36 carbon atoms and of alkyl polyglycosides as described previously, for example the compositions sold under the brand names Montanov™ 68, Montanov™ 14, Montanov™ 82, Montanov™ 202, Montanov™ S, Montanov™ W018, Montanov™ L, Fluidanov™ 20X and Easynov™.

As examples of agents for protecting against the ultraviolet rays of the sun that may be present in formulation (F₂) for topical use that is a subject of the present invention, pigments, organic sunscreens and inorganic sunscreens are denoted.

As pigments used as agents for protecting against the ultraviolet rays of the sun that may be present in formulation (F₂) for topical use that is a subject of the present invention, there are, for example, titanium dioxide, brown iron oxides, yellow iron oxides, black iron oxides or red iron oxides, or else white or colored nacreous pigments such as titanium mica.

As organic sunscreens used as agents for protecting against the ultraviolet rays of the sun that may be present in formulation (F₂) for topical use that is a subject of the present invention, there are, for example:
those of the family of benzoic acid derivatives, such as para-aminobenzoic acids (PABAs), notably monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA, butyl esters of N,N-dimethyl PABA;
those of the family of anthranilic acid derivatives, such as homomenthyl-N-acetyl anthranilate;
those of the family of salicylic acid derivatives, such as amyl salicylate, homomenthyl salicylate, ethylhexyl salicylate, phenyl salicylate, benzyl salicylate, p-isopropanolphenyl salicylate;
those of the family of cinnamic acid derivatives, such as ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy 2-ethoxyethyl cinnamate, p-methoxycyclohexyl cinnamate, ethyl-α-cyano-β-phenyl cinnamate, 2-ethylhexyl-□α-cyano-β-phenyl cinnamate, glyceryl di-para-methoxy mono-2-ethylhexanoyl cinnamate;
those of the family of benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy 4-methoxy benzophenone-5-sulfonate, 4-phenylbenzophenone, 2-ethylhexyl-4′-phenyl benzophenone-2-carboxylate, 2-hydroxy 4-n-octyloxybenzophenone, 4-hydroxy 3-carboxybenzophenone; 3-(4′-methylbenzylidene)-d,l-camphor, 3-(benzylidene)-d,l-camphor, benzalkonium methosulfate camphor; urocanic acid, ethyl urocanate;
those of the family of sulfonic acid derivatives, such as 2-phenylbenzimidazole-5-sulfonic acid and salts thereof; the family of triazine derivatives, such as hydroxyphenyltriazine, ethylhexyloxyhydroxyphenyl-4-methoxyphenyltriazine, 2,4,6-trianillino-(p-carbo-2′-ethylhexyl-1′-oxy)-1,3,5-triazine, benzoic acid 4,4-((6-(((1,1-dimethylethyl)amino)carbonyl)phenyl)amino)-1,3,5-triazine-2,4-diyldiimino)bis(2-ethylhexyl) ester, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; 2-(4-diethylamino-2-hydroxybenzoyl)benzoic acid hexyl ester, 2,4-bis{[4-(2-ethylhexyloxy)-2-hydroxy]phenyl}-6-(4-methoxyphenyl)-1,3,5-triazine, 2,4,6-tris[4-(2-ethylhexyloxycarbonyl)anilino]-1,3,5-triazine, 2-ethylhexyl dimethoxybenzylidene dioxoimidazolidine propionate, the family of diphenylacrylate derivatives, such as 2-ethylhexyl-2-cyano-3,3-diphenyl-2-propenoate, ethyl-2-cyano-3,3-diphenyl-2-propenoate;
those of the family of polysiloxanes, such as benzylidene siloxane malonate.
   As inorganic sunscreens used as agents for protecting against the ultraviolet rays of the sun that may be present in formulation (F₂) for topical use that is a subject of the present invention, there are, for example, titanium oxides, zinc oxides, cerium oxide, zirconium oxide, yellow, red or black iron oxides, and chromium oxides. These mineral screens may or may not be micronized, may or may not have undergone surface treatments and may optionally be in the form of aqueous or oily predispersions.

According to another aspect, a subject of the invention is the use of a formulation (F₂) as defined above, for cleansing the skin, the hair, the scalp or mucous membranes.

The term “for cleansing the skin, the hair, the scalp or mucous membranes” denotes any action intended to enable the removal of soiling present on the skin, the hair, the scalp or mucous membranes of humans or of animals. Examples of soiling present on the skin, the hair, the scalp or mucous membranes of humans or of animals include dusts, soil, sebaceous secretions, sweat, dandruff, dead cells, microorganisms or various chemical substances such as residues of compositions for making up and caring for the skin, the hair, the scalp or mucous membranes.

According to another aspect, a subject of the invention is a process for cleansing the skin, the hair, the scalp and/or mucous membranes, characterized in that it comprises:
at least one step a₂) of applying formulation (F₂) as defined above to the skin, the hair, the scalp or mucous membranes, followed by
at least one step b₂) of rinsing said skin, said hair, said scalp or said mucous membranes treated in step a₂).

In step a₂) of the cleansing process that is the subject of the invention, formulation (F₂) for topical use is applied to the surface of the skin, the hair, the scalp or mucous membranes comprising soiling to be cleansed by any means, for example by application by means of a support consisting of synthetic or natural, woven or nonwoven textile fibers, or paper, preimpregnated with said formulation (F₂) for topical use.

In step b₂) of the cleansing process that is the subject of the invention, the rinsing of the skin, the hair, the scalp or mucous membranes onto which has been applied formulation (F₂) for topical use during step a₂) is performed by spraying with water.

Step b₂) of the cleansing process that is the subject of the invention may be performed at room temperature or at a temperature of between 20° C. and 40° C.

The examples that follow illustrate the invention without, however, limiting it.

### 1) Preparation of Foaming Compositions

### 1.1) Preparation of a Solution of Disodium N-cocoyl Glutamate [Composition (C₁)].

315 kg of water and 177.2 kg of monosodium sodium glutamate monohydrate are placed in a reactor, with stirring and at a temperature of 20° C., followed by 152.5 kg of an aqueous sodium hydroxide solution at 30% by mass so as to reach a pH of 12. 167 kg of cocoyl chloride, which is a mixture of acid chlorides comprising, per 100% by mass, 8% by mass of octanoyl chloride, 8% by mass of decanoyl chloride, 50% by mass of lauroyl chloride, 17% by mass of myristoyl chloride, 8% by mass of palmitoyl chloride, 3% by mass of stearoyl chloride, 4% by mass of oleoyl chloride and 2% by mass of linoleoyl chloride, are then added gradually with stirring, followed by a further 110 kg of the 30% sodium hydroxide solution to keep the pH between 11 and 12. The temperature is maintained between 20° C. and 50° C. for 2 hours.

The mixture obtained is acidified by adding 65 kg of an aqueous sulfuric acid solution at 70% by mass, and is then diluted with 75 kg of water to obtain an aqueous solution of disodium N-cocoyl glutamate [composition (C₁)].

### 1.2) Analytical Features of Composition (C₁) Prepared Previously.

The analytical features of composition (C₁) are collated in table 1 below.

TABLE 1

| | (C₁) |
|---|---|
| Appearance at 20° C. (visual method) | Clear |
| Mass content of water (a) (according to the standard NFT | 56.1% |
| 73-201) | |
| Residual fatty acids (b) (gas chromatography (GC)) | 8.2% |
| pH | 6.3 |
| Sodium chloride content (potentiometric titration (c)) | 4.6% |
| Sodium sulfate content (d) (calculated on feedstock) | 5.6% |
| Mass content of active material (AM₁) (cocoyl glutamate) | 25.5% |
| (AM₁) = 100% − (a) − (b) − (c) − (d) | |

### 2) Preparation of Alkylpolyglycoside-Based Surfactant Compositions

### 2.1) Preparation of a Composition (C₃)

3.7 molar equivalents of a mixture of fatty alcohols (N₁) consisting, per 100% of its mass, of 68% by mass of 1-dodecanol, of 25% by mass of 1-tetradecanol and of 7% by mass of 1-hexadecanol, and then 1 molar equivalent of anhydrous glucose are poured with stirring into a reactor maintained at 80° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed.

After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess of the mixture of fatty alcohols (N₁) is removed by distillation using a thin-film evaporator, and the residue is then diluted in water. After stirring for 30 minutes at 50° C., composition (C₃) is obtained, which comprises 50% by mass of water and 50% by mass of a mixture of alkylpolyglucosides (AM_{APG1}), for which the proportions of alkylpolyglucosides and the mean degree of polymerization of their polyglucoside residue are determined by gas chromatography (GC); it thus comprises, per 100% by mass, 69% by mass of n-dodecyl polyglucosides, 25% by mass of n-tetradecyl polyglucosides and 6% by mass of n-hexadecyl polyglucosides with a degree of polymerization equal to 1.25.

2.2) Preparation of a Composition (C₄) Comprising n-Heptyl Polyglucoside

2.7 molar equivalents of 1-heptanol and then 1 molar equivalent of anhydrous glucose are poured with stirring into a reactor maintained at 40° C., followed by 0.15% by mass of 98% sulfuric acid per 100% by mass of the mixture.

The reaction medium is placed under a partial vacuum of about 0.18×10⁵ Pa (180 mbar) and maintained at 100° C.-105° C. for 4 hours with distillation of the water formed. After cooling to 85° C.-90° C. and neutralizing by addition of 40% sodium hydroxide, the reaction medium thus obtained is discharged at 70° C. and filtered to remove the grains of unreacted glucose.

The filtrate is then poured into another reactor and the excess heptanol is distilled off under partial vacuum, and the residue is then diluted in water.

After stirring for 30 minutes at 50° C., composition (C₄) is obtained comprising 26.4% by mass of water and 73.6% by mass of n-heptyl polyglucosides (AM_{APG2}), with a degree of polymerization, determined by GC, equal to 1.25.

### 3) Preparation of Compositions According to the Invention and of Comparative Compositions

Four compositions, (F₁)_{inv.}, (F₂)_{inv.}, (F₃)_{inv.} and (F₄)_{inv.} according to the invention, and two comparative compositions (F₅)_{comp.} and (F₆)_{comp.} are prepared by pouring, with stirring, into a reactor maintained at 40° C., composition (C₁) and at least one of the compositions (C₃) or (C₄). The mixture is stirred for 30 minutes to obtain one of the compositions (F₁)_{inv.} to (F₄)_{inv.} and (F₅)_{comp.} and (F₆)_{comp.}. The amounts used are collated in table 2 below:

TABLE 2
Amounts used

| | (C₁) | (C₃) | (C₄) |
|---|---|---|---|
| (F₁)_{inv.} | 74.9 g | 11.1 g | 14.0 g |
| (F₂) _{inv.} | 73.8 g | 15.6 g | 10.6 g |
| (F₃) _{inv.} | 72.8 g | 19.9 g | 7.3 g |
| (F₄)_{inv.} | 75.7 g | 8.0 g | 16.4 g |
| (F₅)_{comp.} | 72.1 g | 22.7 g | 5.2 g |
| (F₆)_{comp.} | 76.0 g | 0 g | 24.0 g |

The analytical features of compositions (F₁)_{inv.}, (F₂)_{inv.}, (F₃)_{inv.}, (F₄)_{inv.} and comparative compositions (F₅)_{comp.} (F₆)_{comp.} are collated in table 3 below.

TABLE 3

| | FA⁽¹⁾ | F⁽²⁾ | F′⁽³⁾ | H₂O (%) | Δ₁⁽⁴⁾ | Appearance |
|---|---|---|---|---|---|---|
| (F₁)_{inv.} | 5.84% | 61.4% | 38.6% | 52.32% | 35/65 | Homogeneous |
| (F₂)_{inv.} | 5.76% | 61.8% | 38.2% | 53.04% | 50/50 | Homogeneous |
| (F₃)_{inv.} | 5.68% | 62.2% | 38.8% | 53.74% | 65/35 | Homogeneous |
| (F₄)_{inv.} | 5.90% | 61.1% | 38.9% | 51.85% | 25/75 | Homogeneous |
| (F₅)_{comp.} | 5.62% | 62.4% | 37.6% | 54.18% | 74.8/25.2 | Homogeneous |
| (F₆)_{comp.} | 5.93% | 58.8% | 41.2% | 50.03% | 0/100 | Homogeneous |

⁽¹⁾Residual fatty acids (mass percentage)
⁽²⁾F = (AM₁)/[(AM₁) + (AM_{APG1}) + (AM_{APG2})]
⁽³⁾F′ = [(AM_{APG1}) + (AM_{APG2})]/[(AM₁) + (AM_{APG1}) + (AM_{APG2})]
⁽⁴⁾Δ₁ = (AM_{APG1})/(AM_{APG2})

### 4) Evaluation of the Foaming Properties

### 4.1) Principle of the Evaluation Method

The evaluation of the foaming properties of the test compositions is performed by forming a foam, from a solution of OMS hard water comprising a predetermined mass content of the test compositions, by mechanical stirring at a temperature of 20° C.

### 4.2) Experimental Protocol

250 cm³ aqueous solutions are prepared so as to obtain solutions containing 0.5% by mass of surfactant active material in OMS hard water, from the compositions (F₁)_{inv.}, (F₂)_{inv.}, (F₃)_{inv.}, (F₄)_{inv.}, (F₅)_{comp}., (F₆)_{comp.}, (C₁), (C₃) and (C₄).

250 cm³ aqueous solutions containing 0.39% of surfactant active material in OMS hard water from the compositions (C₁), and also a 250 cm³ aqueous solution containing 0.11% of surfactant active material in OMS hard water from the composition (C₁), are also prepared.

The OMS hard water contains, per liter of permuted water, 0.403 g of anhydrous calcium chloride and 0.139 g of magnesium chloride hexahydrate; which gives it a hardness titer equal to 34° Th.

These solutions are poured into a 500 cm³ beaker and are then stirred using a Rayneri™ laboratory blender (model 33/300) equipped with a butterfly paddle with three hollow arms, at a constant speed of 3000 rpm for 2 minutes.

### 4.3) Expressing the Results

The following parameters are measured for each test:
The expansion time (T_{exp.}): this is the stirring time after which suppression of the vortex in the beaker is observed. Beyond this time, the foam totally surrounds the shaft of the paddle and its level is horizontal;
The half-life time (T_{1/2}): this is the time after which the foam obtained from a certain volume of foaming solution became drained of an amount of solution corresponding to half of the initial volume. For this test, the half-life time is reached when the upper level of the draining water reaches the 125 cm³ mark on the beaker;
The height of foam generated by stirring (Hₜ₀): this is the height of foam generated at the end of the 2 minutes of stirring;
The residual foam height after 30 minutes (Hₜ₃₀): this is the foam height observed 30 minutes after the end of the 2 minutes of stirring.
The difference Δ_{H}=(Hₜ₀−Hₜ₃₀), makes it possible to evaluate comparatively the quality of the foams generated by the various surfactants.
The degree of expansion (T_{F}): this is the value of the ratio between the volume of foam (Vₘ) produced by a foaming composition to the volume (Vₛ) of the foaming solution used (water and emulsifier).

### 4.4) Results Obtained

The results obtained for the aqueous solutions of active material in the OMS hard water for compositions (F₁)_{inv.}, (F₂)_{inv.}, (F₃)_{inv.}, (F₄)_{inv.}, (F₅)_{comp.}, (F₆)_{comp.}, (C₁), (C₃) and (C₄) are indicated in table 4 below.

| 0.50% | (Tₑₓₚ) | | (T_{1/2}) | (Hₜₒ) | (Hₜ₃₀) | (ΔH) | (T_{F}) |
|---|---|---|---|---|---|---|---|
| (F₁)_{inv.} | 12 | s | 48 min | 160 mm | 145 mm | 15 mm | 6.5 |
| (F₂)_{inv.} | 13s | s | 40 min | 170 mm | 145 mm | 25 mm | 6.6 |
| (F₃)_{inv.} | 24s | s | 36 min | 170 mm | 145 mm | 25 mm | 6.5 |
| (F₄)_{inv.} | 10s | s | 46 min | 170 mm | 145 mm | 25 mm | 6.7 |
| (F₅)_{comp.} | 22s | s | 12 min | 175 mm | n.m. | n.m. | n.m. |
| (F₆)_{comp.} | 44s | s | 30 min | 125 mm | 115 mm | 10 mm | 6.8 |
| (C₁) | 27s | s | 37 min | 145 mm | 120 mm | 25 mm | 6.4 |
| (C₃) | >2 | min | n.m. | n.m. | n.m. | n.d. | n.m. |
| (C₄) | >2 | min | n.m. | n.m. | n.m. | n.d. | n.m. |

n.m.: not measurable

### 4.5) Analysis of the Results

These results show that the compositions according to the invention make it possible to prepare foams having all the qualities required for use in cosmetics, unlike the comparative compositions.

### 5) Formulation Examples

The proportions of constituents are expressed as mass percentages.

### 5.1. Foaming Facial Gel

### Formula

Sodium lauryl sulfate: 6%
Composition (F₁)_{inv.} 1%

### Montaline™ C40⁽¹⁾: 5%

### Sepitonic™ M3⁽²⁾: 1%

### Fragrance: 0.1%

Lactic acid: 0.15%

### Water: qs 100%

### Fragrance: 0.3%

### Kathon™ CG (³): 0.08%

Sodium chloride: 0.8%

### 5.2. Bubble Bath

### Formula

Sodium lauryl sulfate: 10%
Composition (F₁)_{inv.} 2.6%

### Fragrance: 0.1%

### Sepicide™ HB⁽⁴⁾: 0.5%

### Capigel™ 98⁽⁵⁾: 4.5%

### Water: qs 100%

### Sepicide™ CI⁽⁸⁾: 0.3%

### Dye: qs

Sodium hydroxide: qs

### 5.3. Liquid Hand Soap

### Formula

Sodium lauryl sulfate: 10%
Composition (F₁)_{inv.} 2.6%

### Amonyl™ 675 SB⁽⁶⁾: 10%

### Fragrance: 0.3%

### Sepicide™HB⁽⁴⁾: 0.5%

### Sepicide™ CI⁽⁸⁾: 0.3%

### Water: qs 100%

Sodium chloride: qs

### 5.4. Facial Cleansing Goam

### Formula

Sodium lauryl sulfate: 5%
Composition (F₁)_{inv.}: 2.9%

### Sepicide™HB⁽⁴⁾: 0.5%

### Fragrance: 0.2%

### Water: qs 100%

### Sepicide™CI⁽⁸⁾: 0.3%

### Sepitonic™ M3⁽²⁾: 1%

### Tromethamine: qs

### Dye: qs

### 5.5. Anti-stress Shampoo

### Formula

Sodium lauryl sulfate: 20%
Composition (F₁)_{inv.} 11.6%

### Sepicide™HB^{( 4)}: 0.5%

### Sepicide™CI⁽⁸⁾: 0.3%

### Fragrance: 0.2%

### Water: qs 100%

### Sepicap™MP⁽⁷⁾: 1%

### Water: 10%

### Capigel™ 98⁽⁵⁾: 3%

Sodium hydroxide: qs pH=7.2

### Dye: qs

(1): Montaline™ C40 is the salt cocamidopropyl betainamide monoethanolamine chloride.
(2): Sepitonic™ M3, mixture of magnesium aspartate, zinc gluconate and copper gluconate, is an energizing active agent sold by the company SEPPIC.
(3): Kathon™ CG, mixture of methylchloroisothiazolinone and methylisothiazolinone, is a preserving agent sold by the company Röhm & Haas.
(4): Sepicide™ HB, a mixture of phenoxyethanol, methyl paraben, ethyl paraben, propyl paraben and butyl paraben, is a preserving agent sold by the company SEPPIC.
(5): Capigel™ 98 is an acrylate copolymer sold by the company SEPPIC.
(6): Amonyl™ 675SB is a cocoamidopropyl hydroxysultaine, sold by the company SEPPIC.
(7): Sepicap™ MP is a mixture of N-cocoyl amino acids and of the potassium salt of panthenyl phosphate dimethicone PEG-7, sold by the company SEPPIC.
(8): Sepicide™ CI, imidazolidinyl urea, is a preserving agent sold by the company SEPPIC.

1. A process for improving the foaming properties of a cleansing formulation for topical use (F₁), comprising, per 100% of its mass, from 0.5% to 10% by mass of a composition (C₁), said composition (C₁) comprising, per 100% of its mass:
(α)—from 65% to 90% by mass of at least one compound of formula (I):
R₁—C(═O)—NH—CH(COOH)—(CH₂)₂—COOH (I)
in partially or totally salified acid form, in which the group R₁—C(═O)— represents a linear or branched, saturated or unsaturated acyl radical including from 8 to 18 carbon atoms, and
(β)—from 10% to 35% by mass of at least one compound of formula (II):
R₁—C(═O)—OH (II)
in partially or totally salified acid form, in which the group R₁ is as defined for formula (I);
said comprising a step a1) of incorporating into said formulation (F₁) an effective amount of at least one composition (C₂) comprising, per 100% of its mass:
(γ)—from 14% to 80% by mass of a composition (C₃) or of a mixture of compositions (C₃), said composition (C₃) being represented by formula (III):
R₃—O-(G₃)ₚ-H (III)
in which R₃ represents a linear or branched, saturated or unsaturated aliphatic radical including from 12 to 16 carbon atoms, G₃ represents a reducing sugar residue and p represents a decimal number greater than or equal to 1.05 and less than or equal to 5, said composition (C₃) consisting of a mixture of compounds represented by formulae (III₁), (III₂), (III₃), (III₄) and (III₅):
R₃—O-(G₃)₁-H (III₁),
R₃—O-(G₃)₂-H (III₂),
R₃—O-(G₃)₃-H (III₃),
R₃—O-(G₃)₄-H (III₄),
R₃—O-(G₃)₅-H (III₅),
in the respective molar proportions a₁, a₂, a₃, a₄ and a₅ such that: the sum a₁+a₂+a₃+a₄+a₅ is equal to 1, and
the sum a₁+2a₂+3a₃+4a₄+5a₅ is equal to p;
(5)—from 0% to 3% by mass of at least one alcohol of formula (IV):
R₃—OH (IV)
in which R₃ is as defined for the preceding formula (III),
(ε)—from 20% to 80% of a composition (C₄) or of a mixture of compositions (C₄), said composition (C₄) being represented by formula (V):
R₄—O -(G₄)_{q}-H (V)
in which R₄ represents a linear aliphatic radical chosen from butyl, pentyl, hexyl and heptyl radicals, G₄ represents a reducing sugar residue and q represents a decimal number greater than or equal to 1.05 and less than or equal to 5, said composition (C₄) consisting of a mixture of compounds represented by formulae (V₁), (V₂), (V₃), (V₄) and (V₅):
R₄—O-(G₄)₁-H (V₁),
R₄—O-(G₄)₂-H (V₂),
R₄—O-(G₄)₃-H (V₃),
R₄—O-(G₄)₄-H (V₄),
R₄—O-(G₄)₅-H (V₅),
in the respective molar proportions a′₁, a′₂, a′₃, a′₄ and a′₅, such that: the sum a′₁+a′₂+a′₃+a′₄+a′₅ is equal to 1, and the sum a′₁+2a′₂+3a′₃+4a′₄+5a′₅ is equal to q; and
(η)—from 0% to 3% by mass of at least one alcohol of formula (VI):
R₄—OH (VI)
in which R₄ is as defined for the preceding formula (V).

2. The process as defined in claim 1, wherein, in formulae (I) and (II), the group R₁—C(═O)— represents an acyl radical chosen from octanoyl, decanoyl, dodecanoyl, tetradecanoyl, hexadecanoyl, octadecanoyl, 9-octadecenoyl, 9,12-octadecadienoyl and 9,12,15-octadecatrienoyl radicals.

3. The process as defined in claim 1, wherein said composition (C₂) comprises, for 100% of its mass:
(γ)—a mass proportion of said composition (C₃) of greater than or equal to 14% and less than 70%, and
(δ)—a mass proportion of said alcohol of formula (IV) of greater than or equal to 0% and less than or equal to 3%,
(ε)—a mass proportion of said composition (C₄) of greater than or equal to 30% and less than or equal to 80%, and
(η)—a mass proportion of said alcohol of formula (VI) of greater than or equal to 0% and less than or equal to 3%.

4. The process as defined in claim 1, wherein, in formula (III), G₃ represents a reducing sugar residue chosen from glucose, xylose and arabinose residues.

5. The process as defined in claim 1, wherein, in formula (III), p represents a decimal number greater than or equal to 1.05 and less than or equal to 2.5.

6. The process as defined in claim 1, wherein, in formulae (III) and (IV), R₃ represents a linear alkyl radical chosen from dodecyl (n-C₁₂H₂₅—), tetradecyl (n-C₁₄H₂₉—) and hexadecyl (n-C₁₆H₃₃—) radicals.

7. The process as defined in claim 1, wherein, in formula (V), G₄ represents a reducing sugar residue chosen from glucose, xylose and arabinose residues.

8. The process as defined in claim 1, wherein, in formula (V), q represents a decimal number greater than or equal to 1.05 and less than or equal to 2.5.

9. The process as defined in claim 1, wherein, in formulae (V) and (VI), R₄ represents a linear alkyl radical chosen from hexyl (n-C₆H₁₃—) and heptyl (n-C₇H₁₅—) radicals.

10. The process as defined in claim 1, wherein said composition (C₂) comprises a mixture of compositions (C₃) and of compositions (C₄), said mixture comprising, per 100% of mass:
(γ1)—from 13.6% to 44.4% by mass of a composition (C₃) represented by formula (III) in which R₃ represents the dodecyl radical (n-C₁₂H₂₅—),
(γ₂)—from 5% to 16.25% by mass of a composition (C₃) represented by formula (III) in which R₃ represents the tetradecyl radical (n-C₁₄H₂₉—) and
(ε₁)—from 1.4% to 4.55% by mass of a composition (C₃) represented by formula (III) in which R₃ represents the hexadecyl radical (n-C₁₆H₃₃—),
(ε₁)—from 35% to 80% by mass of a composition (C₄) represented by formula (V) in which R₄ represents the heptyl radical (n-C₇H₁₅—).

11. The process as defined in claim 1, wherein the mass ratio:
Δ=Mass of compound(s) of formula (I)/[Mass of composition (C₃) Mass of composition (C₄)], is greater than or equal to 20/80 and less than or equal to 65/35.

12. The process as defined in claim 1, wherein the mass ratio:
Δ₁=Mass of composition (C₃)/Mass of composition (C₄) is greater than or equal to 20/80 and less than or equal to 70/30.

13. A formulation (F₂) for topical use comprising, per 100% of mass:
a)—from 0.5% to 10% by mass of a composition (C₁), said composition (C₁) comprising, per 100% of its mass:
(α)—from 65% to 90% by mass of at least one compound of formula (I):
R₁—C(═O)—NH—CH(COOH)—(CH₂)₂—COOH (I)
in partially or totally salified acid form, in which the group R₁—C(═O)— represents a linear or branched, saturated or unsaturated acyl radical including from 8 to 18 carbon atoms, and
(β)—from 10% to 35% by mass of at least one compound of formula (II):
R₁—C(═O)—OH (II)
in partially or totally salified acid form, in which the group R₁ is as defined for formula (I),
b)—from 0.1% to 10% by mass of at least one composition (C₂) as defined in claim 1, and
c)—from 3.4% to 20% by mass of at least one foaming and/or detergent surfactant chosen from anionic and amphoteric foaming and/or detergent surfactants, and
d)—from 60% to 96% by mass of water.

14. (canceled)

15. A process for cleansing the skin, the hair, the scalp and/or mucous membranes, the process comprising:
at least one step a₂) of applying formulation (F₂) as defined in claim 13 to the skin, the hair, the scalp or mucous membranes, followed by
at least one step b₂) of rinsing said skin, said hair, said scalp or said mucous membranes treated in step a₂).

16. The process as defined in claim 1, wherein said composition (C₂) comprises, for 100% of its mass:
(γ)—a mass proportion of said composition (C₃) of greater than or equal to 14% and less than 70%, and
(δ)—a mass proportion of said alcohol of formula (IV) of greater than or equal to 0% and less than or equal to 3%,
(ε)—a mass proportion of said composition (C₄) of greater than or equal to 30% and less than or equal to 80%, and
(η)—a mass proportion of said alcohol of formula (VI) of greater than or equal to 0% and less than or equal to 3%.

17. The process as defined in claim 2, wherein, in formula (III), G₃ represents a reducing sugar residue chosen from glucose, xylose and arabinose residues.

18. The process as defined in claim 3, wherein, in formula (III), G₃ represents a reducing sugar residue chosen from glucose, xylose and arabinose residues.

19. The process as defined in claim 2, wherein, in formula (III), p represents a decimal number greater than or equal to 1.05 and less than or equal to 2.5.

20. The process as defined in claim 3, wherein, in formula (III), p represents a decimal number greater than or equal to 1.05 and less than or equal to 2.5.

21. The process as defined in claim 4, wherein, in formula (III), p represents a decimal number greater than or equal to 1.05 and less than or equal to 2.5.
